(19) United States
(12) Patent Application Publication
(10) Pub. No.: US 2008/0260690 A1
(43) Pub. Date: Oct. 23, 2008

(54) Interferon in Influenza

(75) Inventor: Giampiero De Luca, Conches (CH)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: ARES TRADING S.A., Aubonne (CH)

(21) Appl. No.: 12/091,985

(22) PCT Filed: Nov. 16, 2006
(86) PCT No.: PCT/EP2006/068588

§ 371 Date: Apr. 29, 2008

Related U.S. Application Data
(60) Provisional application No. 60/738,483, filed on Nov. 21, 2005.

(30) Foreign Application Priority Data
Nov. 18, 2005 (EP) 05110965.0

(51) Int. Cl. A61K 38/21; A61P 37/00

(52) U.S. Cl. 424/85.6; 424/85.4

(57) ABSTRACT

The use of an interferon (IFN) for the manufacture of a medicament for treatment and/or prevention of influenza, preferably avian influenza, is described in the present invention.

### FIELD OF THE INVENTION

The present invention relates to the use of an interferon (IFN) for the manufacture of a medicament for treatment and/or prevention of influenza.

### BACKGROUND OF THE INVENTION

Influenza is a common infectious disease in humans, which is caused by influenza virus. Influenza virus is transmitted very easily by aerosols from infected people. The incubation time is 24-72 hours. The characteristic symptoms are a rapid onset of fever, together with cough, sore throat, as well as muscle pain, arthralgy and malaise. Additional symptoms can also be observed, like pharyngitis, conjonctivitis, bronchitis, diarrhoea or vomiting, but they are less frequent. Usually, patients recover within 1 and 2 weeks. However, a feeling of weakness can last for several weeks. Complications caused by influenza virus can be a respiratory distress, pneumonia caused by the influenza virus or by bacteria (staphylococcus, streptococcus, pneumococcus, Haemophilus influenzae).

Avian influenza is an infectious disease of birds caused by type A strains of the influenza virus. The disease, which was first identified in Italy more than 100 years ago, occurs worldwide and may infect humans.

### DESCRIPTION OF THE INVENTION

The main object of the present invention is the use of an interferon (IFN) alone or in combination with an antiviral agent for the manufacture of a medicament for treatment and/or prevention of Influenza.

Influenza viruses belong to the family of orthomyxoviruses. Three different types can be distinguished: influenza A, influenza B and influenza C viruses. The last one seems not to be associated with severe illness. The most severe symptoms are associated with influenza A viruses. Many sub-types of influenza A viruses are known and they are classified according to the origin of the hemagglutinin and of the neuraminidase: 15 types of hemagglutinin and 9 neuraminidase have been described. Influenza A subtypes still circulating in the population are influenza A (H3N2) appeared in 1968, and influenza A (H1N1) reappeared in 1977.

More recently, 2 new influenza viruses of avian origin could be detected in humans. It was an influenza A (H5N1) and an influenza A (H9N2) strain. The first one caused the death of 6 persons in Hong Kong. The virus was directly transmitted from chickens to humans. The second one was detected in 2 hospitalized children Hong Kong. Patients showed weak symptoms and could be discharged from the hospital without any further problems.

Fifteen subtypes of influenza virus are known to infect birds, thus providing an extensive reservoir of influenza viruses potentially circulating in bird populations. To date, all outbreaks of the highly pathogenic form have been caused by influenza A viruses of subtypes H5 and H7. Recent research has shown that viruses of low pathogenicity can, after circulation for sometimes short periods in a poultry population, mutate into highly pathogenic viruses. During a 1983-1984 epidemic in the United States of America, the H5N2 virus initially caused low mortality, but within six months became highly pathogenic, with a mortality approaching 90%. During a 1999-2001 epidemic in Italy, the H7N1 virus, initially of low pathogenicity, mutated within 9 months to a highly pathogenic form.

Two other avian influenza viruses have recently caused illness in humans. An outbreak of highly pathogenic H7N7 avian influenza, which began in the Netherlands in February 2003, caused the death of one veterinarian two months later, and mild illness in 83 other humans. Mild cases of avian influenza H9N2 in children occurred in Hong Kong in 1999 (two cases) and in mid-December 2003 (one case). H9N2 is not highly pathogenic in birds. The most recent cause for alarm occurred in January 2004, when laboratory tests confirmed the presence of H5N1 avian influenza virus in human cases of severe respiratory disease in the northern part of Viet Nam.

Of the 15 avian influenza virus subtypes, H5N1 is of particular concern for several reasons. H5N1 mutates rapidly and has a documented propensity to acquire genes from viruses infecting other animal species. Its ability to cause severe disease in humans has now been documented on two occasions. In addition, laboratory studies have demonstrated that isolates from this virus have a high pathogenicity and can cause severe disease in humans. Birds that survive infection excrete virus for at least 10 days, orally and in faeces, thus facilitating further spread at live poultry markets and by migratory birds.

Antiviral drugs, some of which can be used for both treatment and prevention, are clinically effective against influenza A virus strains in otherwise healthy adults and children, but have some limitations. Some of these drugs are also expensive and supplies are limited.

Accordingly in one aspect the present invention provides for the use of an interferon (IFN) or an isoform, mutein, fused protein, functional derivative, active fraction or salt thereof, for the manufacture of a medicament for treatment and/or prevention of influenza.

In another aspect the present invention provides for the use of an interferon (IFN) or an isoform, mutein, fused protein, functional derivative, active fraction or salt thereof for treatment and/or prevention of influenza.

In another aspect the present invention provides for an interferon (IFN) or an isoform, mutein, fused protein, functional derivative, active fraction or salt thereof for use in the treatment and/or prevention of influenza.

The isoform, mutein, fused protein, functional derivative, active fraction or salt of an interferon (IFN) may also collectively be called interferon (IFN) variants hereinbelow.

Such uses according to the invention of an interferon or an interferon variant include monotherapy, i.e. the interferon or the interferon variant is used as the only antiviral compound administered to the patient (monotherapy). In an alternative embodiment the interferon or the interferon variant is administered in addition to, or together with, an antiviral agent as further defined hereinbelow (combination therapy).

In a further aspect the present invention for the use of an IFN (IFN), or an isoform, mutein, fused protein, functional derivative, active fraction or salt thereof, in combination with an antiviral agent for the manufacture of a medicament for treatment and/or prevention of influenza for simultaneous, sequential or separate use.

In a preferred embodiment the influenza is avian influenza. For example the avian influenza may be caused by a type A strain or a type B strain of influenza virus. In a preferred embodiment the avian influenza is caused by a type A strain of influenza virus.

In further preferred embodiments the avian influenza is caused by a influenza virus of subtype H5, H7, or H9. In further preferred embodiments the avian influenza is caused by a influenza virus of any of the subtypes H5N2, H7N1, H7N7, H9N2, or H5N1. In a particularly preferred embodiment the subtype is H5N1.

There is also provided a method of treating a patient having an infection with avian influenza, wherein such patient is administered a therapeutically effective amount of an interferon (IFN) or an isoform, mutein, fused protein, functional derivative, active fraction or salt thereof. In one embodiment the interferon (IFN) or an isoform, mutein, fused protein, functional derivative, active fraction or salt thereof is the only antiviral compound administered to the patient (monotherapy), whereas, in an alternative embodiment the interferon (IFN) or an isoform, mutein, fused protein, functional derivative, active fraction or salt thereof is administered in addition to, or together with, an antiviral agent as further defined hereinbelow (combination therapy).

The antiviral agent may be selected from the group of neuraminidase inhibitors, such as Oseltamivir (Tamiflu®) and Zanamivir (Relenza®), adamantanes, such as Amantadine (Symmetrel®) and Rimantadine (Flumadine®), or Ribavirin (Rebetol®). In a particularly preferred embodiment the antiviral agent is Ribavirin.

There is also provided a method of prophylaxis of a subject at risk for an infection with avian influenza, wherein such patient is administered a therapeutically effective amount of an interferon (IFN) or an isoform, mutein, fused protein, functional derivative, active fraction or salt thereof. In one embodiment the interferon (IFN) or an isoform, mutein, fused protein, functional derivative, active fraction or salt thereof is the only antiviral compound administered to the patient (monotherapy), whereas, in an alternative embodiment the interferon (IFN) or an isoform, mutein, fused protein, functional derivative, active fraction or salt thereof is administered in addition to, or together with, an antiviral agent as further defined hereinbelow (combination therapy).

In a particularly preferred embodiment the IFN is recombinant human IFN-beta. In one preferred embodiment the recombinant human IFN-beta has a CHO cell-derived glycosylation.

In another embodiment the IFN is consensus interferon.

In another embodiment the IFN is a long-acting interferon-beta, such as for example a fused protein comprising an immunoglobulin (Ig) fragment, preferably the Fc portion of an immunoglobulin. For example, the long-acting interferon-beta may be selected from pegylated interferon-beta or interferon-beta Fc-fusion proteins.

The IFN may be administered at a dosage of about 1 to 50 μg per person per day, or about 10 to 30 μg per person per day or about 10 to 20 μg per person per day.

The IFN may, for example, be administered daily or every other day.

Also, the IFN may, for example, be administered twice or three times per week.

In one embodiment the IFN may, for example, be administered subcutaneously. Alternatively, the IFN may, for example, be administered intramuscularly. Furthermore the IFN may be delivered by a spray device.

In one preferred embodiment the IFN is delivered within less than 3, preferably less than 2 days after infection with an influenza virus.

In another preferred embodiment the IFN is dosed at least at 44 mcg s.c. per administration.

In another preferred embodiment the IFN is administered at least 3× weekly.

In another preferred embodiment the antiviral agent is administered at a dosage of about 100 to 2000 mg per person per day, or about 400 to 1200 mg per person per day, or about 800 to 1000 mg per person per day, or about 1000 to 1200 mg per person per day.

Interferons are cytokines, i.e. soluble proteins that transmit messages between cells and play an essential role in the immune system by helping to destroy microorganisms that cause infection and repairing any resulting damage. Interferons are naturally secreted by infected cells and were first identified in 1957. Their name is derived from the fact that they “interfere” with viral replication and production.

Interferons exhibit both antiviral and antiproliferative activity. On the basis of biochemical and immunological properties, the naturally-occurring human interferons are grouped into three major classes: interferon-alpha (leukocyte), interferon-beta (fibroblast) and interferon-gamma (immune). Alpha-interferon is currently approved in the United States and other countries for the treatment of hairy cell leukemia, venereal warts, Kaposi's Sarcoma (a cancer commonly afflicting patients suffering from Acquired Immune Deficiency Syndrome (AIDS)), and chronic non-A, non-B hepatitis.

Further, interferons (IFNs) are glycoproteins produced by the body in response to a viral infection. They inhibit the multiplication of viruses in protected cells. Consisting of a lower molecular weight protein, IFNs are remarkably non-specific in their action, i.e. IFN induced by one virus is effective against a broad range of other viruses. They are however species-specific, i.e. IFN produced by one species will only stimulate antiviral activity in cells of the same or a closely related species. IFNs were the first group of cytokines to be exploited for their potential anti-tumor and antiviral activities.

The three major IFNs are referred to as IFN-α, IFN-β and IFN-γ. Such main kinds of IFNs were initially classified according to their cells of origin (leukocyte, fibroblast or T cell). However, it became clear that several types might be produced by one cell. Hence leukocyte IFN is now called IFN-α, fibroblast IFN is IFN-β and T cell IFN is IFN-γ. There is also a fourth type of IFN, lymphoblastoid IFN, produced in the “Namalwa” cell line (derived from Burkitt's lymphoma), which seems to produce a mixture of both leukocyte and fibroblast IFN.

The interferon unit or International unit for interferon (U or IU, for international unit) has been reported as a measure of IFN activity defined as the amount necessary to protect 50% of the cells against viral damage. The assay that may be used to measure bioactivity is the cytopathic effect inhibition assay as described (Rubinstein, et al. 1981; Familletti, P. C., et al., 1981). In this antiviral assay for interferon about 1 unit/ml of interferon is the quantity necessary to produce a cytopathic effect of 50%. The units are determined with respect to the international reference standard for Hu-IFN-beta provided by the National Institutes of Health (Pestka, S. 1986).

Every class of IFN contains several distinct types. IFN-β and IFN-γ are each the product of a single gene.

The proteins classified as IFNs-α are the most diverse group, containing about 15 types. There is a cluster of IFN-α genes on chromosome 9, containing at least 23 members, of which 15 are active and transcribed. Mature IFNs-α are not glycosylated.

IFNs-α and IFN-β are all the same length (165 or 166 amino acids) with similar biological activities. IFNs-γ are 146 amino acids in length, and resemble the α and β classes less closely. Only IFNs-γ can activate macrophages or induce the maturation of killer T cells. These new types of therapeutic agents can are sometimes called biologic response modifiers (BRMs), because they have an effect on the response of the organism to the tumor, affecting recognition via immunomodulation.

Human fibroblast interferon (IFN-β) has antiviral activity and can also stimulate natural killer cells against neoplastic cells. It is a polypeptide of about 20,000 Da induced by viruses and double-stranded RNAs. From the nucleotide sequence of the gene for fibroblast interferon, cloned by recombinant DNA technology, (Derynk et al. 1980) deduced the complete amino acid sequence of the protein. It is 166 amino acid long.

Shepard et al. (1981) described a mutation at base 842 (Cys→Tyr at position 141) that abolished its anti-viral activity, and a variant clone with a deletion of nucleotides 1119-1121.

Mark et al. (1984) inserted an artificial mutation by replacing base 469 (T) with (A) causing an amino acid switch from Cys→Ser at position 17. The resulting IFN-β was reported to be as active as the ‘native’ IFN-β and stable during long-term storage (−70° C.).

Rebif® (recombinant human interferon-β), the latest development in interferon therapy for multiple sclerosis (MS), is interferon(IFN)-beta 1a, produced from mammalian cell lines.

The treatment of SRS with interferons alone or in combination with other anti-viral agents has not yet been reported in the literature.

The term “treatment” within the context of this invention refers to any beneficial effect on progression of disease, including attenuation, reduction, decrease or diminishing of the pathological development after onset of disease.

An “interferon” or “IFN”, as used herein, is intended to include any molecule defined as such in the literature. In particular, IFN-α, IFN-β and IFN-γ are included in the above definition. IFN-β is the preferred IFN according to the present invention. IFN-β suitable in accordance with the present invention is commercially available e.g. as Rebif® (Serono), Avonex® (Biogen) or Betaferon® (Schering). The use of interferons of human origin is also preferred in accordance with the present invention. The term interferon, as used herein, is intended to encompass salts, functional derivatives, variants, analogs and active fragments thereof.

The term “interferon-beta (IFN-β)”, as used herein, is intended to include fibroblast interferon in particular of human origin, as obtained by isolation from biological fluids or as obtained by DNA recombinant techniques from prokaryotic or eukaryotic host cells, as well as its salts, functional derivatives, variants, analogs and active fragments.

As used herein the term “muteins” refers to analogs of IFN in which one or more of the amino acid residues of a natural IFN are replaced by different amino acid residues, or are deleted, or one or more amino acid residues are added to the natural sequence of IFN, without changing considerably the activity of the resulting products as compared to the wild type IFN. These muteins are prepared by known synthesis and/or by site-directed mutagenesis techniques, or any other known technique suitable therefore. Preferred muteins include e.g. the ones described by Shepard et al. (1981) or Market al. (1984).

Any such mutein preferably has a sequence of amino acids sufficiently duplicative of that of IFN, such as to have substantially similar or even better activity to an IFN. The biological function of interferon is well known to the person skilled in the art, and biological standards are established and available e.g. from the National Institute for Biological Standards and Control (http://immunology.org/links/NIBSC).

Bioassays for the determination of IFN activity have been described. An IFN assay may for example be carried out as described by Rubinstein et al., 1981. Thus, it can be determined whether any given mutein has substantially a similar, or even a better, activity than IFN by means of routine experimentation.

Muteins of IFN, which can be used in accordance with the present invention, or nucleic acid coding therefore, include a finite set of substantially corresponding sequences as substitution peptides or polynucleotides which can be routinely obtained by one of ordinary skill in the art, without undue experimentation, based on the teachings and guidance presented herein.

Preferred changes for muteins in accordance with the present invention are what are known as “conservative” substitutions. Conservative amino acid substitutions of polypeptides or proteins of the invention, may include synonymous amino acids within a group, which have sufficiently similar physicochemical properties that substitution between members of the group will preserve the biological function of the molecule. It is clear that insertions and deletions of amino acids may also be made in the above-defined sequences without altering their function, particularly if the insertions or deletions only involve a few amino acids, e.g., under thirty, and preferably under ten, and do not remove or displace amino acids which are critical to a functional conformation, e.g., cysteine residues. Proteins and muteins produced by such deletions and/or insertions come within the purview of the present invention.

Preferably, the synonymous amino acid groups are those defined in Table I. More preferably, the synonymous amino acid groups are those defined in Table II; and most preferably the synonymous amino acid groups are those defined in Table III.

TABLE I
Preferred Groups of Synonymous Amino Acids

| Amino Acid | Synonymous Group |
|---|---|
| Ser | Ser, Thr, Gly, Asn |
| Arg | Arg, Gln, Lys, Glu, His |
| Leu | Ile, Phe, Tyr, Met, Val, Leu |
| Pro | Gly, Ala, Thr, Pro |
| Thr | Pro, Ser, Ala, Gly, His, Gln, Thr |
| Ala | Gly, Thr, Pro, Ala |
| Val | Met, Tyr, Phe, Ile, Leu, Val |
| Gly | Ala, Thr, Pro, Ser, Gly |
| Ile | Met, Tyr, Phe, Val, Leu, Ile |
| Phe | Trp, Met, Tyr, Ile, Val, Leu, Phe |
| Tyr | Trp, Met, Phe, Ile, Val, Leu, Tyr |
| Cys | Ser, Thr, Cys |
| His | Glu, Lys, Gln, Thr, Arg, His |
| Gln | Glu, Lys, Asn, His, Thr, Arg, Gln |
| Asn | Gln, Asp, Ser, Asn |
| Lys | Glu, Gln, His, Arg, Lys |
| Asp | Glu, Asn, Asp |
| Glu | Asp, Lys, Asn, Gln, His, Arg, Glu |
| Met | Phe, Ile, Val, Leu, Met |
| Trp | Trp |

TABLE II
More Preferred Groups of Synonymous Amino Acids

| Amino Acid | Synonymous Group |
|---|---|
| Ser | Ser |
| Arg | His, Lys, Arg |
| Leu | Leu, Ile, Phe, Met |
| Pro | Ala, Pro |
| Thr | Thr |
| Ala | Pro, Ala |
| Val | Val, Met, Ile |
| Gly | Gly |
| Ile | Ile, Met, Phe, Val, Leu |
| Phe | Met, Tyr, Ile, Leu, Phe |
| Tyr | Phe, Tyr |
| Cys | Cys, Ser |
| His | His, Gln, Arg |
| Gln | Glu, Gln, His |
| Asn | Asp, Asn |
| Lys | Lys, Arg |
| Asp | Asp, Asn |
| Glu | Glu, Gln |
| Met | Met, Phe, Ile, Val, Leu |
| Trp | Trp |

TABLE III
Most Preferred Groups of Synonymous Amino Acids

| Amino Acid | Synonymous Group |
|---|---|
| Ser | Ser |
| Arg | Arg |
| Leu | Leu, Ile, Met |
| Pro | Pro |
| Thr | Thr |
| Ala | Ala |
| Val | Val |
| Gly | Gly |
| Ile | Ile, Met, Leu |
| Phe | Phe |
| Tyr | Tyr |
| Cys | Cys, Ser |
| His | His |
| Gln | Gln |
| Asn | Asn |
| Lys | Lys |
| Asp | Asp |
| Glu | Glu |
| Met | Met, Ile, Leu |
| Trp | Met |

Examples of production of amino acid substitutions in proteins which can be used for obtaining muteins of IFN, for use in the present invention include any known method steps, such as presented in U.S. Pat. Nos. 4,959,314, 4,588,585 and 4,737,462, to Mark et al; 5,116,943 to Koths et al., 4,965,195 to Namen et al; 4,879,111 to Chong et al; and 5,017,691 to Lee et al; and lysine substituted proteins presented in U.S. Pat. No. 4,904,584 (Shaw et al). Specific muteins of IFN-beta have been described, for example by Mark et al., 1984.

The term “fused protein” refers to a polypeptide comprising an IFN, or a mutein thereof, fused to another protein, which e.g., has an extended residence time in body fluids. An IFN may thus be fused to another protein, polypeptide or the like, e.g., an immunoglobulin or a fragment thereof.

“Functional derivatives” as used herein cover derivatives of IFN, and their muteins and fused proteins, which may be prepared from the functional groups which occur as side chains on the residues or the N- or C-terminal groups, by means known in the art, and are included in the invention as long as they remain pharmaceutically acceptable, i.e. they do not destroy the activity of the protein which is substantially similar to the activity IFN, and do not confer toxic properties on compositions containing it. These derivatives may, for example, include polyethylene glycol side-chains, which may mask antigenic sites and extend the residence of IFN in body fluids. Other derivatives include aliphatic esters of the carboxyl groups, amides of the carboxyl groups by reaction with ammonia or with primary or secondary amines, N-acyl derivatives of free amino groups of the amino acid residues formed with acyl moieties (e.g. alkanoyl or carbocyclic aroyl groups) or O-acyl derivatives of free hydroxyl groups (for example that of seryl or threonyl residues) formed with acyl moieties.

As “active fractions” of IFN, or muteins and fused proteins, the present invention covers any fragment or precursors of the polypeptide chain of the protein molecule alone or together with associated molecules or residues linked thereto, e.g., sugar or phosphate residues, or aggregates of the protein molecule or the sugar residues by themselves, provided said fraction has no significantly reduced activity as compared to the corresponding IFN.

The term “salts” herein refers to both salts of carboxyl groups and to acid addition salts of amino groups of the proteins described above or analogs thereof. Salts of a carboxyl group may be formed by means known in the art and include inorganic salts, for example, sodium, calcium, ammonium, ferric or zinc salts, and the like, and salts with organic bases as those formed, for example, with amines, such as triethanolamine, arginine or lysine, piperidine, procaine and the like. Acid addition salts include, for example, salts with mineral acids, such as, for example, hydrochloric acid or sulfuric acid, and salts with organic acids, such as, for example, acetic acid or oxalic acid. Of course, any such salts must retain the biological activity of the proteins (IFN) relevant to the present invention, i.e., the ability to bind to the corresponding receptor and initiate receptor signaling.

According to one embodiment of the invention, R¹ of formula (I) is H. In another embodiment R² is OH. In a further embodiment A is N. In yet a further embodiment of the invention R³ and R⁵ form a 6-membered heterocyclic ring. In a preferred embodiment of the invention, the heterocyclic ring is a pyrimidine or a pyrimidine-one.

In accordance with the present invention, antiviral can be used incombination with an interferon to potentiate its beneficial effects. According to the present invention, the use of Ribavirin (1-β-D-ribofuranosyl-1H-1,2,4-Triazole-3-carboxamide), as antiviral is especially preferred.

In accordance with the present invention, the use of recombinant human IFN-beta and the compounds of the invention is further particularly preferred.

A special kind of interferon variant has been described recently. The so-called “consensus interferons” are non-naturally occurring variants of IFN (U.S. Pat. No. 6,013,253). According to a preferred embodiment of the invention, the compounds of the invention are used in combination with a consensus interferon.

As used herein, human interferon consensus (IFN-con) shall mean a non-naturally-occurring polypeptide, which predominantly includes those amino acid residues that are common to a subset of IFN-alpha's representative of the majority of the naturally-occurring human leukocyte interferon subtype sequences and which includes, at one or more of those positions where there is no amino acid common to all subtypes, an amino acid which predominantly occurs at that position and in no event includes any amino acid residue which is not existent in that position in at least one naturally-occurring subtype. IFN-con encompasses but is not limited to the amino acid sequences designated IFN-con1, IFN-con2 and IFN-con3 which are disclosed in U.S. Pat. Nos. 4,695,623, 4,897,471 and 5,541,293. DNA sequences encoding IFN-con may be produced as described in the above-mentioned patents, or by other standard methods.

In a further preferred embodiment, the fused protein comprises an Ig fusion. The fusion may be direct, or via a short linker peptide which can be as short as 1 to 3 amino acid residues in length or longer, for example, 13 amino acid residues in length. Said linker may be a tripeptide of the sequence E-F-M (Glu-Phe-Met), for example, or a 13-amino acid linker sequence comprising Glu-Phe-Gly-Ala-Gly-Leu-Val-Leu-Gly-Gly-Gln-Phe-Met introduced between the sequence of IFN and the immunoglobulin sequence. The resulting fusion protein may have improved properties, such as an extended residence time in body fluids (half-life), increased specific activity, increased expression level, or the purification of the fusion protein is facilitated.

In a further preferred embodiment, IFN is fused to the constant region of an Ig molecule. Preferably, it is fused to heavy chain regions, like the CH2 and CH3 domains of human IgG1, for example. Other isoforms of Ig molecules are also suitable for the generation of fusion proteins according to the present invention, such as isoforms IgG₂, IgG₃ or IgG₄, or other Ig classes, like IgM or IgA, for example. Fusion proteins may be monomeric or multimeric, hetero- or homomultimeric.

In a further preferred embodiment, the functional derivative comprises at least one moiety attached to one or more functional groups, which occur as one or more side chains on the amino acid residues. Preferably, the moiety is a polyethylene (PEG) moiety. PEGylation may be carried out by known methods, such as the ones described in WO99/55377, for example.

The dosage administered, as single or multiple doses, to an individual will vary depending upon a variety of factors, including pharmacokinetic properties, the route of administration, patient conditions and characteristics (sex, age, body weight, health, size), extent of symptoms, concurrent treatments, frequency of treatment and the effect desired.

Standard dosages of human IFN-beta range from 80 000 IU/kg and 200 000 IU/kg per day or 6 MIU (million international units) and 12 MIU per person per day or 22 to 44 μg (microgram) per person. In accordance with the present invention, IFN may preferably be administered at a dosage of about 1 to 50 μg, more preferably of about 10 to 30 μg or about 10 to 20 μg per person per day.

The administration of active ingredients in accordance with the present invention may be by intravenous, intramuscular or subcutaneous route. The preferred route of administration for IFN is the subcutaneous route.

IFN may also be administered daily or every other day, of less frequent. Preferably, IFN is administered one, twice or three times per week

The preferred route of administration is subcutaneous administration, administered e.g. three times a week. A further preferred route of administration is the intramuscular administration, which may e.g. be applied once a week.

Preferably 22 to 44 μg or 6 MIU to 12 MIU of IFN-beta is administered three times a week by subcutaneous injection.

IFN-beta may be administered subcutaneously, at a dosage of 250 to 300 μg or 8 MIU to 9.6 MIU, every other day.

30 μg or 6 MIU IFN-beta may further be administered intramuscularly once a week.

In a preferred embodiment Ribavirin is administered in combination with IFN-beta and it is administered at a dosage of about 100 to 2000 mg per person per day, preferably of about 400 to 1200 mg per person per day, more preferably about 800 to 1000 mg per person per day, or about 1000 to 1200 mg per person per day. For patients weighing less than 65 kg the usual dose is 800 mg per day, for patients weighing 65 to 85 kg the usual dose is 1000 mg per day and for patients weighting more than 85 kg the usual dose is 1200 mg per day. The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

In a preferred embodiment, Ribavirin is administered orally.

Ribavirin may be administered by injection or, preferably, orally. Depending on the mode of administration, the compound can be formulated with the appropriate diluents and carriers to form ointments, creams, foams, and solutions having from about 0.01% to about 15% by weight, preferably from about 1% to about 10% by weight of the compound. For injection, Ribavirin is in the form of a solution or suspension, dissolved or suspended in physiologically compatible solution from about 10 mg/ml to about 1500 mg/ml. Injection may be intravenous, intermuscular, intracerebral, subcutaneous, or intraperitoneal.

For oral administration, Ribavirin may be in capsule, tablet, oral suspension, or syrup form. The tablet or capsules may contain from about 10 to 500 mg of Ribavirin. Preferably they may contain about 300 mg of Ribavirin. The capsules may be the usual gelatin capsules and may contain, in addition to the Ribavirin in the quantity indicated above, a small quantity, for example less than 5% by weight, magnesium stearate or other excipient. Tablets may contain the foregoing amount of the compound and a binder, which may be a gelatin solution, a starch paste in water, polyvinyl pyrilidone, polyvinyl alcohol in water, etc. with a typical sugar coating.

The compounds of the invention and IFN may be formulated in a pharmaceutical composition.

The term “pharmaceutically acceptable” is meant to encompass any carrier, which does not interfere with effectiveness of the biological activity of the active ingredient and that is not toxic to the host to which it is administered. For example, for parenteral administration, the active protein(s) may be formulated in a unit dosage form for injection in vehicles such as saline, dextrose solution, serum albumin and Ringer's solution.

The active ingredients of the pharmaceutical composition according to the invention can be administered to an individual in a variety of ways. The routes of administration include intradermal, transdermal (e.g. in slow release formulations), intramuscular, intraperitoneal, intravenous, subcutaneous, oral, epidural, topical, and intranasal routes. Any other therapeutically efficacious route of administration can be used, for example absorption through epithelial or endothelial tissues or by gene therapy wherein a DNA molecule encoding the active agent is administered to the patient (e.g. via a vector), which causes the active agent to be expressed and secreted in vivo. In addition, the protein(s) according to the invention can be administered together with other components of biologically active agents such as pharmaceutically acceptable surfactants, excipients, carriers, diluents and vehicles.

The subcutaneous route is preferred in accordance with the present invention.

Another possibility of carrying out the present invention is to activate endogenously the genes for IFN. In this case, a vector for inducing and/or enhancing the endogenous production of IFN in a cell normally silent for expression of IFN, or which expresses amounts of IFN which are not sufficient, are is used for treatment of influenza. The vector may comprise regulatory sequences functional in the cells desired to express IFN. Such regulatory sequences may be promoters or enhancers, for example. The regulatory sequence may then be introduced into the right locus of the genome by homologous recombination, thus operably linking the regulatory sequence with the gene, the expression of which is required to be induced or enhanced. The technology is usually referred to as “endogenous gene activation” (EGA), and it is described e.g. in WO 91/09955.

The invention further relates to the use of a cell that has been genetically modified to produce IFN in the manufacture of a medicament for the treatment and/or prevention of influenza.

For parenteral (e.g. intravenous, subcutaneous, intramuscular) administration, IFN can be formulated as a solution, suspension, emulsion or lyophilised powder in association with a pharmaceutically acceptable parenteral vehicle (e.g. water, saline, dextrose solution) and additives that maintain isotonicity (e.g. mannitol) or chemical stability (e.g. preservatives and buffers). The formulation is sterilized by commonly used techniques.

According to the invention, the compounds of the invention and IFN can be administered prophylactically or therapeutically to an individual prior to, simultaneously or sequentially with other therapeutic regimens or agents (e.g. multiple drug regimens), in a therapeutically effective amount. Active agents that are administered simultaneously with other therapeutic agents can be administered in the same or different compositions.

All references cited herein, including journal articles or abstracts, published or unpublished U.S. or foreign patent application, issued U.S. or foreign patents or any other references, are entirely incorporated by reference herein, including all data, tables, figures and text presented in the cited references. Additionally, the entire contents of the references cited within the references cited herein are also entirely incorporated by reference.

Reference to known method steps, conventional methods steps, known methods or conventional methods is not any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various application such specific embodiments, without undue experimentation, without departing from the general concept of the present invention. Therefore, such adaptations and modifications are intended to be within the meaning of a range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

### EXAMPLE 1

The efficacy of IFN is studied in an experimental influenza virus infection model in the mouse. In this model, intranasal inoculation of the virus causes severe haemorrhagic pneumonia which leads to the death of the animals within 7 to 10 days of infection. The experimental design envisages evaluation of the therapeutic efficacy of the study substance, as assessed on the basis of survival of the infected animals. To this end, IFN is administered to the animals at various doses, on a daily basis for 7 days, starting from a few hours after infection.

Preferably an avian influenza strain is used, such as in particular an H5N1 strain.

Interferon (IFN), preferably recombinant IFN-beta, is tested as a monotherapy as well as in combination with antiviral agents such as neuraminidase inhibitors, such as Oseltamivir (Tamiflu®) and Zanamivir (Relenza®), adamantanes, such as Amantadine (Symmetrel®) and Rimantadine (Flumadine®), or Ribavirin (Rebetol®)

A reduction in the mortality of treated animals is observed.

### EXAMPLE 2

Four-week-old female inbred Balb/c AnCrIBR mice are used. A suitable IFN preparation is administered to the animals via the intraperitoneal route at various times after infection with the influenza virus. The IFN concentrations are chosen so as to obtain a range of doses in the animals' blood similar to the effective range in vitro.

The mice are inoculated intranasally (i.n.) with a suspension containing the influenza virus A/PR at a multiplicity of infection of 2 HAU/mouse, after light anaesthesia with ether. On the basis of previous experimental data, the influenza virus at this multiplicity of infection produces haemorrhagic pneumonia that leads to the death of 80% of the animals by one week after infection. For the purposes of monitoring the infection trend, both virological and immunological parameters are monitored in addition to studying survival curves.

As a virological parameter, the viral load is determined. At different times after infection, the lungs of infected and control mice are taken as samples, weighed and homogenised in RPMI containing antibiotics. After centrifuging, the supernatants are suitably diluted and the viral load is analysed by means of the CPE-50% test. On the basis of this method, confluent MDCK cells are infected with the supernatants serially diluted in RPMI added with antibiotics at 2% FCS and incubated for three days at 37° C. in a 5% CO2 atmosphere. For each dilution, the wells showing positive effects are counted and compared with those showing negative cytopathic effects according to the Reed and Muench formula. The CPE-50% titre is calculated in units/ml.

As an immunological parameter, levels of inflammatory cytokines are evaluated using the ELISA method. A 96-well plate is used for the experiment. The plate is coated with monoclonal antibodies to the cytokines to be studied, incubated overnight at 4° C. Later, 200 μl/well of 1% BSA in carbonate buffer were added for 30 min at 37° C. Washings are then done with 0.25% TBS+Tween 20 and the samples are added for 4 hours at 37° C. As a reference curve recombinant cytokines in scalar dilution are used. Washings are then performed and an anti-cytokine polyclonal antibody, different from the first one, is added and left overnight at +4° C. Later, to washings with 0.5% TBS+Tween 20, MgCl2 2 nM the third antibody conjugated to the anzyme alkaline phosphatase is added for 4 h at 37° C. Lastly, a substrate for the enzyme (100 μl/well) is added and the readout is taken using the ELISA reader and a 405 nm filter. The following antibodies are analysed: 1) monoclonal rat anti-mouse TNF-alpha/recombinant mouse IL-6; 2) recombinant mouse TNF-alpha/recombinant mouse IL-6; 3) polyclonal rabbit anti-mouse TNF-alpha/polyclonal goat anti-mouse IL-6; 4) goat anti-rabbit IgG-alkaline phosphatase/anti-goat IgG alkaline phoshatase.

Preferably an avian influenza strain is used, such as in particular an H5N1 strain.

Interferon (IFN), preferably recombinant IFN-beta, is tested as a monotherapy as well as in combination with antiviral agents such as neuraminidase inhibitors, such as Oseltamivir (Tamiflu®) and Zanamivir (Relenza®), adamantanes, such as Amantadine (Symmetrel®) and Rimantadine (Flumadine®), or Ribavirin (Rebetol®)

A reduction in the mortality of treated animals is observed.

### REFERENCES

1. Derynk R. et al., Nature 1980; 285, 542-547.
2. Familletti, P. C., Rubinstein, S., and Pestka, S. 1981 “A Convenient and Rapid Cytopathic Effect Inhibition Assay for Interferon,” in Methods in Enzymology, Vol. 78 (S. Pestka, ed.), Academic Press, New York, 387-394;
3. Mark D. F. et al., Proc. Natl. Acad. Sci. U.S.A., 81 (18) 5662-5666 (1984).
4. Pestka, S. (1986) “Interferon Standards and General Abbreviations, in Methods in Enzymology (S. Pestka, ed.), Academic Press, New York 119, 14-23.
5. Rubinstein, S., Familletti, P. C., and Pestka, S. Convenient Assay for Interferons. J. Virol 1981; 37, 755-758.
6. Shepard H. M. et al., Nature 1981; 294, 563-565.

1-27. (canceled)

28. A method of treating influenza comprising the administration of interferon (IFN) or an isoform, mutein or fused protein thereof to an individual in need of treatment.

29. The method according to claim 28, wherein the influenza is avian influenza.

30. The method according to claim 29, wherein the avian influenza is caused by a type A strain or a type B strain of influenza virus.

31. The method according to claim 30, wherein the avian influenza is caused by a type A strain of influenza virus.

32. The method according to claim 29, wherein the avian influenza is caused by an influenza virus of subtype H5, H7, or H9.

33. The method according to claim 32, wherein the avian influenza is caused by an influenza virus of any of the subtypes H5N2, H7N1, H7N7, H9N2, or H5N1.

34. The method according to claim 33, wherein the avian influenza is caused by an influenza virus of the subtype H5N1.

35. The method according to claim 28, further comprising the administration of an antiviral agent selected from Oseltamivir (Tamiflu®), Zanamivir (Relenza®), adamantanes, Amantadine (Symmetrel®), Rimantadine (Flumadine®) or Ribavirin (Rebetol®).

36. The method according to claim 35, wherein said antiviral agent is Ribavirin.

37. The method according to claim 28, wherein said IFN is recombinant human IFN-beta, recombinant human IFN-beta which has a CHO cell-derived glycosylation, or consensus interferon.

38. The method according to claim 28, wherein said interferon is pegylated interferon-beta or interferon-beta Fc-fusion proteins.

39. The method according to claim 28, wherein said IFN is administered at a dosage of about 1 to 50 μg per person per day, or about 10 to 30 μg per person per day or about 10 to 20 μg per person per day.

40. The method according to claim 28, wherein said IFN is administered:
a) daily or every other day;
b) twice or three times per week; or
c) at least three times weekly.

41. The method according to claim 28, wherein said IFN is administered:
a) subcutaneously;
b) intramuscularly; or
c) by a spray device.

42. The method according to claim 28, wherein said IFN is administered within less than 3 days after infection with an influenza virus.

43. The method according to claim 28, wherein the IFN at is dosed at least at 44 mcg s.c. per administration.

44. The method according to claim 35, wherein the antiviral agent is administered at a dosage of about 100 to 2000 mg per person per day, or about 400 to 1200 mg per person per day, or about 800 to 1000 mg per person per day, or about 1000 to 1200 mg per person per day.

45. The method according to claim 35, wherein said Ribavirin is administered orally.

46. A method of prophylaxis of influenza infection comprising the administration of interferon (IFN) or an isoform, mutein or fused protein thereof to an individual not yet exposed to influenza virus.
